(19) United States
(12) Patent Application Publication
(10) Pub. No.: US 2021/0085262 A1
(43) Pub. Date: Mar. 25, 2021

(54) ACCOMPANYING CARDIAC INSUFFICIENCY PATIENTS

(71) Applicant: Bayer Aktiengesellschaft, Leverkusen (DE)

(72) Inventors: Frank KRAMER, Wülfrath (DE); Wilfried DINH, Rhede (DE)

(73) Assignee: Bayer Aktiengesellschaft, Leverkusen (DE)

(21) Appl. No.: 16/961,872

(22) PCT Filed: Jan. 9, 2019
(86) PCT No.: PCT/EP2019/050374

§ 371 Date: Jul. 13, 2020

(30) Foreign Application Priority Data
Jan. 16, 2018 (EP) 18151940.6

(51) Int. Cl. A61B 5/00; A61B 5/11; A61B 5/08; A61B 5/01; A61B 5/024

(52) U.S. Cl. CPC A61B 5/742; A61B 5/7285; A61B 5/11; A61B 5/0022; A61B 5/01; A61B 5/4875; A61B 5/024; A61B 5/08

(57) ABSTRACT

The present invention relates to the field of accompanying patients and alleviating the symptoms of a disease, particularly in patients suffering from cardiac insufficiency. The invention relates to a method and a system for monitoring the state of health of a large number of patients.

### CROSS REFERENCE TO RELATED APPLICATIONS

This application is a national stage application under 35 U.S.C. § 371 of International Application No. PCT/EP2019/050374, filed internationally on Jan. 9, 2019, which claims the benefit of European Application No. 18151940.6, filed Jan. 16, 2018.

### FIELD

The present invention relates generally to accompanying patients in improving the symptoms of a disease, especially patients who are suffering from a heart failure, and in particular, systems methods for monitoring the health status of a multiplicity of patients.

### BACKGROUND

Chronic heart failure is the most common diagnosis in internal medicine in developed industrial countries; about one to two percent of the adult population suffer from heart failure. The prevalence increases disproportionately with increase in age.

Pathophysiologically, heart failure is defined as a state in which the heart is not capable of supplying the metabolically active tissue with sufficient blood (and hence with oxygen)—despite normal filling pressures or only at the expense of increased filling pressures. From a clinical perspective, heart failure is described as a syndrome in which the patients exhibit typical symptoms (e.g., respiratory distress, ankle edemas, exhaustion) and clinical signs (such as, for example, jugular vein congestion, moist crackles via the lungs, displaced apex beat) and these changes are caused by a structural or functional abnormality of the heart.

There are numerous causes of heart failure, which vary distinctly in different regions of the world. A generally accepted classification of the causes of heart failure does not exist, and this is undoubtedly because there are some overlaps between the individual categories.

Approximately half of all heart-failure patients have a reduced left-ventricular ejection fraction, a situation which is called heart failure with reduced ejection fraction (HFrEF). With regard to pathophysiology and treatment, HFrEF is the best-understood form of heart failure. Around two thirds of patients have a coronary heart disease. Other common causes are arterial hypertension and diabetes mellitus, which can both cause a heart failure either primarily or via the path of coronary heart disease.

Heart failure with preserved ejection fraction (HFpEF) has a different etiological profile. Typical HFpEF patients are older, often female and overweight. They increasingly suffer from hypertension and atrial fibrillation. Diagnosing HFpEF is sometimes complex and is often carried out by means of exclusion methods.

Drugs which are effective in HFrEF therapy and improve the prognosis (ACE inhibitors, AT1 receptor blockers, beta blockers) have no significant effect on morbidity and mortality in HFpEF patients.

Other reasons for a heart failure are a virus infection, which remains undetected in many cases, chemotherapy, arrhythmias, and familial and genetic cardiomyopathies.

### BRIEF DESCRIPTION OF THE DRAWINGS

The present disclosure will now be described, by way of example only, with reference to the accompanying drawings, in which:

FIG. 1 shows a system for monitoring a plurality of persons who are suffering from heart failure, according to some embodiments;

FIG. 2 shows a system for monitoring a plurality of persons who are suffering from heart failure, according to some embodiments; and

FIG. 3 shows a system for monitoring a plurality of persons who are suffering from heart failure from the perspective of a patient.

### DETAILED DESCRIPTION

The prognosis of heart failure is altogether poor. It is particularly patients who do not receive optimal therapy that die with heart failure relatively frequently. If hospitalization occurs because of a deterioration in heart failure, this has a particularly drastic effect on the prognosis: the 30-day death rate is ten percent. After 60 days, 30 to 50 percent of these patients are rehospitalized or dead; after one year, 30 percent of these patients are dead.

It would therefore be desirable to be able to identify a heart failure in patients early, to be able to identify the cause and nature of the heart failure early, to be able to identify an improvement and/or deterioration in the state of a patient with a heart failure early and/or to be able to accompany a patient with heart failure even beyond a hospital in order to avoid a rehospitalization and/or more severe consequences.

This is achieved by the present invention.

In a first aspect, the present invention provides a system comprising
sensors for monitoring physiological parameters of a person,
a self-assessment unit,
a data integrations unit,
a data synchronization unit,
a data memory and
a laboratory data acquisition unit,

the sensors, the self-assessment unit and the laboratory data acquisition unit being connected to the data integration unit via a network,

the sensors, the self-assessment unit and the laboratory data acquisition unit being configured such that they transmit data to the data integration unit via the network,

the data integration unit being configured such that it receives data from the sensors, the self-assessment unit and the laboratory data acquisition unit, links them together on the basis of the person and transmits them to a data synchronization unit,

the data synchronization unit being configured such that it receives data from the data integration unit, temporally synchronizes the received data and transmits them to the data memory,

the data memory being configured such that it stores the temporally synchronized data.

Method comprising the steps of
monitoring physiological parameters of a person by means of sensors, the sensors permanently capturing sensor data,
ascertaining self-assessment data from the person,
ascertaining laboratory data relating to the person,
transmitting the sensor data, the self-assessment data and the laboratory data to a data integration unit via a network,
linking the sensor data, self-assessment data and laboratory data on the basis of the person by means of the data integration unit,
transmitting the linked data to a data synchronization unit,
temporally synchronizing the linked sensor data, self-assessment data and laboratory data by means of the data synchronization unit,
transmitting the temporally synchronized data to a data memory,
storing the temporally synchronized data.

The present invention makes it possible to accompany a patient with a heart failure or suspected heart failure even beyond a hospital. Despite the permanent monitoring of the health status of the patient, patient activity is minimally restricted. By means of the present invention, improvements and deteriorations in the health status of the patient are promptly identified, meaning that measures can be taken immediately in the event of any deterioration. Moreover, therapy can be optimal tailored to the patient.

The invention will be more particularly elucidated below without distinguishing between the subjects of the invention (system, method). On the contrary, the following elucidations are intended to apply analogously to all the subjects of the invention, irrespective of in which context they occur.

If steps are stated in an order in the present description or in the claims, this does not necessarily mean that the invention is restricted to the stated order. On the contrary, it is conceivable that the steps can also be executed in a different order or else in parallel to one another, unless one step builds upon another step, this absolutely requiring that the building step be executed subsequently (this being, however, clear in the individual case). The stated orders are thus preferred embodiments of the method according to the invention.

The focus of the present invention is on a person—also referred to as a patient in this description—whose health status is monitored by bringing together, linking together and temporally synchronizing various data sources providing information about the physiological state and the well-being of the person.

Preferred data sources are sensors for capturing one or more physiological parameters, one or more laboratory data acquisition units, one or more self-assessment units and/or one or more drug-intake monitoring units.

The system according to the invention is designed such that it can monitor a multiplicity of persons/patients simultaneously. To be able to link data from various data sources and assign the data to a plurality of individuals, an unambiguous (individual) identifier is preferably used for each person/patient. Such an identifier can be an alphanumerical code. Each person receives an individual code, by means of which data can be unambiguously assigned to the person. The data acquired according to the invention contain the unambiguous identifier in, for example, the so-called header. Header refers to additional information (metadata) supplementing the payload at the start of a block of data; the additional information can be used to describe the processing of the data (e.g., the data format, the address information of a data packet to be transported or the character coding used) or to characterize the data (affiliation to a particular person).

According to the invention, at least the following data are used:
sensor data
laboratory data
self-assessment data.

Further data can be used, such as, for example, data from a medical examination (diagnosis data, therapy data, etc.) and/or data relating to the intake of one or more drugs.

Sensors are used for capturing sensor data.

A “sensor” is a technical component which can capture certain physical or chemical properties and/or the material nature of its environment in a qualitative manner or in a quantitative manner as a measurement variable. The properties are captured by means of physical or chemical effects and transformed into further-processable, usually electrical or optical signals.

Sensors are used for automatically capturing physiological parameters.

The term “physiological parameter” is understood to mean a measurable variable which provides information about the physical and biochemical state and/or the physical and biochemical processes in the cells, tissues and organs of an organism. Examples of physiological parameters are: body weight, body temperature, heart rate, heart rhythm, (arterial) blood pressure, skin conductance, tremor (frequency), electrolyte/protein concentration and composition in body fluids, standard laboratory parameters, visual acuity, activity of specific brain areas, electrical activities of cardiac muscle fibers (e.g., captured by means of an electrocardiograph), central venous pressure, arterial oxygen saturation, respiratory rate (to name but just a few). The effects in the body of a patient that are caused in combination with drugs or caused by drugs (pharmacokinetics, pharmacodynamics) and the actions of medical devices on the body of a patient are to be covered by the term “physiological parameters”, too.

The capturing of the sensor data by means of sensors is done especially in order to monitor a few physiological properties of the patient.

“Monitoring” means that measurement values are permanently automatically captured.

“Automatically” means that a sensor which has been activated captures measurement values for a period generally lasting longer than one day, preferably longer than one week, without a further intervention by a person. Generally, an individual measurement of a physiological property by means of a sensor requires a certain time span. The term “permanently” means that the sensor carries out a multiplicity of individual measurements over a monitoring period generally stretching over several hours to days or weeks, the time interval between two successive individual measurements being sufficiently small for a continuous development of the measured variable over time to be identifiable (in contrast to larger time intervals, which merely represent snapshots that do not, however, make it possible to draw any conclusions about the continuous temporal profile).

Sensors which a person wears on the body (e.g., as a so-called “wearable”) or in the body (e.g., as a so-called “implantable”) in a continuous manner (at least during the monitoring period) are used. The sensors are thus usable in a transportable and mobile manner.

The sensors capture physiological parameters which provide information about the health status of the patient or indicate an improvement and/or deterioration in the health status. The sensors can directly measure physiological parameters; however, it is also conceivable that one or more sensors measure one variable or measure multiple variables, from which it is possible to ascertain one or more physiological parameters by mathematical calculations and, where applicable, a calibration.

In this connection, an important physiological parameter is the electrocardiogram (ECG for short). An electrocardiogram is the temporal profile of the sum of electrical activities of all cardiac muscle fibers, captured by means of an electrocardiograph. Each contraction of cardiac muscle is preceded by an electrical stimulation which normally originates from the sinus node. Via the electrical conduction system of the heart that is composed of specialized cardiac muscle cells, it travels to the other cardiac muscle cells. These electrical voltage changes on the heart can, for example, be measured on the body surface and recorded over the course of time. The result is a recurring picture of the electrical cardiac cycle.

A typical ECG recording consists of five identifiable deflections. Each deflection is named using one of the letters P, Q, R, S, T. P refers to the first function of a recording and represents the depolarization of the atria of the heart. The next function is composed of the deflections Q, R and S. It represents the depolarization of the ventricles. The deflection representing the repolarization of the atria is usually not detectable because of the strength of the QRS function. The last function is T, which represents the repolarization of the ventricles.

From the ECG, it is possible to determine heart rate, heart rhythm and the electrical axis of the heart (cf. Cabrera system) and to read the electrical activity of atria and ventricles. For the diagnosis of cardiac arrhythmias such as extra beats (extrasystoles) and disorders in electrical conduction and propagation (e.g., bundle branch block and AV block), the ECG is just as indispensable as it is for identifying a myocardial ischemia or a myocardial infarction. Disorders in repolarization can lead to so-called ST-T changes (changes in the ST segment or the T wave).

The ECG can also provide indications of a thickening of the heart wall (myocaridal hypertrophy), an abnormal load on the right or left side of the heart, inflammations of the pericardium (pericarditis) or cardiac muscle (myocarditis) and also electrolyte disorders and adverse drug effects.

The sensor for monitoring the electrocardiogram can be an implantable sensor. An example of such a commercially available sensor is the implantable heart monitoring system “Reveal LINQ” from Medtronic GmbH.

However, it can also be a sensor which is worn on the body. Essentially, a distinction can be made between two types of wearable heart rate sensors that are commercially available as “wearables”: ECG heart rate sensors and PPG heart rate sensors. In the case of the ECG heart rate sensors, the heart rate is measured on the skin of a person on the basis of an electrocardiographic signal caused by the heartbeat. The signal is measured by means of electrodes which are in contact with the body at at least two points. Details on measuring the electrocardiographic signal can, for example, be found in: Arthur C. Guyton: Human Physiology and Mechanisms of Disease, 3^{rd} Edition, W.B. Saunders Company, 1982, ISBN 4-7557-0072-8, chapter 13: The Electrocardiogram. In the case of professional diagnostic systems in the area of medical technology, up to ten electrodes are often attached to the chest and to the ribs of a patient. ECG signals of such diagnostic systems provide precise information in relation to the various components (P, QRS and T signal paths) of a heartbeat. In sport, unipolar ECG heart rate sensors are usually used. They involve measuring heart activity using a chest belt containing two electrodes. Such a sensor is, for example, described in patent specification U.S. Pat. No. 6,775,566B2. Commercially, such heart rate sensors are, for example, sold under the names H7 or H10 by Polar Electro GmbH Deutschland. A comparatively new trend for measuring heart rate is photoplethysmography (PPG). The method takes advantage of the fact that the amount of blood that is transported in the arteries changes with the cardiac cycle. PPG heart rate sensors are, for example, described in EP1579802A1 and US2014276119.

A further variable which can be sensor-captured is physical activity. This is preferably ascertained by motion sensors. Preferably, a so-called activity tracker having acceleration sensors and gyroscope sensors is used. Acceleration sensors measure the linear movement of the sensor in all three planes in space, while the gyroscope sensors capture the rotation in all three planes in space. By combining both measurement values (movement and rotation), it is possible to capture the movements executed, with algorithms ascertaining from the measurement values the nature of the movement executed by a person wearing a relevant activity sensor. Preferably, the activity sensor additionally has an altimeter (e.g., a barometric altimeter, which measures the air pressure and calculates the altitude therefrom) in order to capture, for example, the climbing of steps.

Activity sensors are commercially available in diverse form, for example in the form of so-called fitness armbands or smartwatches. Preferably, an activity sensor which is worn on the trunk of the body is used; one example is the MoveMonitor from McRoberts B. V.

A further variable which can be sensor-captured is respiratory rate. Respiratory rate refers to the number of breaths per unit of time, which is usually specified in breaths per minute. One breath comprises an inhalation and an exhalation.

There are respiratory rate sensors which are worn as a chest or abdominal belt by a person (for example, MindMedia respiratory sensors). There are respiratory rate sensors which are worn on the neck by a person (for example, Masimo respiratory rate sensors). A multiplicity of different respiratory rate sensors designed as “wearables” is provided by Vandrico. Many implantable heart rate sensors and/or pacemakers as well have the option of capturing thorax movement (and hence respiratory rate) by means of impedance measurement.

A further variable which can be sensor-captured is body temperature. Generally, this means the temperature of the interior of the body, the core body temperature. Sensors for monitoring body temperature are described in the prior art.

A further variable which can be sensor-captured is body fluid status. Monitoring has the goal of determining the water content of the body, especially in the region of the chest. In a preferred embodiment, the body fluid status (water content) is measured via (transthoracic) impedance. Sensors for measuring transthoracic impedance are commercially available. There are implantable sensors and sensors in which band electrodes are worn on the skin. Details on measuring transthoracic impedance and determining the water content in the region of the chest are described in the literature (see, for example: F. Amberger, St. Jude Medical GmbH: Therapie der Herzinsuffizienz durch aktive Implantate—Status and Perspektiven [Heart failure therapy through active implants—status and perspectives], KARDIOTECHNIK 3/2011, pages 77 to 81; A. Fein et al.: Evaluation of Transthoracic Electrical Impedance in the Diagnosis of Pulmonary Edema, http://assets.fluke.com/BiomedDocs/PPP085_Impedance_Educational.ppt; Alberto García Lledó et al.: SYSTEM FOR MEASURING THE TRANSTHORACIC ELECTRICAL IMPEDANCE TO THE ECG SIGNAL, INTERNATIONAL CONGRESS ON COMPUTATIONAL BIOENGINEERING; M. Doblaré, M. Cerrolaza and H. Rodrigues (Eds.), España, 2003; W. H. Tand et al: Measuring impedance in congestive heart failure: Current options and clinical applications, Am. Heart. J. 2009 March; 157(3): 402-411). However, it is also conceivable to use other sensors for determining water content (see, for example: Julian Lenk: Methodenvergleich zur Messung der Körperzusammensetzung bei Patienten mit chronischer Herzinsuffizienz [Comparison of methods for measuring body composition in chronic heart failure patients], thesis for attainment of the academic degree Doctor medicinae submitted to the medical faculty of Charité-Universitätsmedizin Berlin, doctorate obtained on: May 30, 2015).

Preferably, one sensor unit which can capture multiple variables in parallel is used for sensor monitoring of physiological parameters. For example, the AVIVO™ Mobile Patient Management (MPM) System from Medtronic Inc. is capable of monitoring the electrocardiogram, respiratory rate, body temperature, respiratory rate and body fluid status.

The sensor-captured sensor data are usually transmitted wirelessly (e.g., via radio communication) or nonwirelessly from the corresponding sensors to one or more computer units. Such a computer unit can also be used for controlling one or more sensors. Such a computer unit can acquire the (raw) data, process them (e.g., noise suppression, mean value formation, digitalization, conversion, integration, differentiation, transformation and the like) and transmit them to the data integration unit according to the invention via a network (e.g., cellular network and/or Internet). The computer unit used can, for example, be a tablet computer or a smartphone or a smartwatch. A computer unit which receives data from one or more sensors and transmits them (after a processing step if necessary) to the data integration unit is also referred to as a sensor unit in this description.

Besides the physiological parameters which are permanently sensor-captured, what are captured are further physiological parameters which change less rapidly over time than the stated sensor-captured parameters and/or for which wearable and mobile sensors are not (yet) available for achieving permanent monitoring and/or for which permanent monitoring is too complicated and/or too expensive and/or for which permanent data acquisition would lead to an inappropriate restriction of the patient and/or for which permanent sensor-capturing is out of the question for other/further reasons.

Usually, this concerns laboratory data which are obtained in a laboratory by analysis of body fluids, body excretion products and/or other samples (e.g., tissue samples) from the patient. What can also be concerned are data which are obtained by examinations by medical specialists, such as, for example, radiological images (projectional radiography, X-ray computed tomography), sonography images, magnetic resonance imaging images and the like.

A laboratory data acquisition unit is used for acquiring said laboratory data. For example, it is conceivable that a physician or medical employee inputs the results of laboratory investigations into a laboratory computer or that the results are automatically saved on a data memory of the laboratory computer. It is conceivable that the laboratory computer is connected via a network to the data integration unit according to the invention, which retrieves laboratory data from the data memory of the laboratory computer. Also conceivable is that a physician or medical employee delivers the results of laboratory investigations to the data integration unit directly via a network connection via an interface (e.g., a Web interface).

Besides the objectively acquired physiological data, the well-being of the patient also plays an important role in the monitoring of health. Subjective feeling can also make a considerable contribution to the understanding of the objectively acquired physiological data and of the correlation between various data. If it is captured by sensors that a person has experienced a physical strain, for example because the respiratory rate and the heart rate have risen, this may be because just low levels of physical exertion in everyday life place a strain on the person; however, another possibility is that the person consciously and gladly brought about the situation of physical strain, for example as part of a sporting activity. A self-assessment can provide clarity here about the causes of physiological features.

For example, the issue of self-assessment plays an important role in clinical studies as well. In the English-language literature, the term “Patient Reported Outcomes” (abbreviation: PRO) is used as an umbrella term for many different concepts for measuring subjectively felt health statuses. The common basis of said concepts is that patient status is personally assessed and reported by the patient.

Subjective feeling is collected by use of a self-assessment unit, with the aid of which the patient can record information about subjective health status. Preference is given to a list of questions which are to be answered by a patient. Preferably, the questions are answered with the aid of a computer (e.g., a tablet computer or a smartphone). One possibility is that the patient has questions displayed on a screen and/or read out via a speaker. One possibility is that the patient inputs the questions into the computer by inputting text via an input device (e.g., keyboard, mouse, touchscreen and/or a microphone (by means of speech input)). It is conceivable that a chatbot is used in order to facilitate the input of all items of information for the patient.

It is conceivable that the questions are recurring questions which are to be answered once or more than once a day by a patient. It is conceivable that some of the questions are asked in response to a defined event. It is, for example, conceivable that it is captured by means of a sensor that a physiological parameter is outside a defined range (e.g., an increased respiratory rate is established). As a response to this event, the patient can, for example, receive a message via his/her smartphone or a smartwatch or the like that a defined event has occurred and that said patient should please answer one or more questions, for example in order to find out the causes and/or the accompanying circumstances in relation to the event.

The questions can be of a psychometric nature and/or preference-based. At the heart of the psychometric approach is the description of the external, internal and anticipated experiences of the individual, by the individual. Said experiences can be based on the presence, the frequency and the intensity of symptoms, behaviors, capabilities or feelings of the individual questioned. The preference-based approach measures the value which patients assign to a health status.

An example of a questionnaire for collecting the self-assessment of a patient who is suffering from heart failure is the “Kansas City Cardiomyopathy Questionnaire” (KCCQ; see, for example, Faller H. et al., Psychother Psych Med 2005, 55, 200-208).

The self-assessment unit is configured such that it transmits the collected self-assessment data to the data integration unit. The transmission can be initiated by the patient (e.g., by the patient pressing a (virtual) “Send button” after answering the questionnaire). Also conceivable is that each answer by a patient is immediately transmitted to the data integration unit. Also conceivable is that the self-assessment data are transmitted to the data integration unit at defined time points. Mixed forms and variations of the procedures presented here are likewise conceivable.

The intake of drugs can, too, be captured by the system according to the invention. “Drug” refers to a substance or a mixture of substances that exerts a therapeutic effect. A term synonymous with the term “drug” is the term “medicament”.

The term “intake” is not to be understood to have a limited meaning of only an oral administration of a drug. On the contrary, any conceivable form of administration is to be covered by the term “intake”, such as, for example, aural, buccal, inhalational, intra-arterial, intra-articular, intragluteal, intracutaneous, intramuscular, intraocular, intrauterine, intravenous, intravitreal, intranasal, percutaneous, rectal, sublingual, subcutaneous, topical, transdermal, vaginal and the like. Preferably, the drug is personally taken by the patient; i.e., a medical specialist is not required for administering the drug to the patient. Usually, the drug is present in the form of defined portions, from which a patient is to take a defined quantity (one portion, two portions, half a portion or the like) at defined time points or within defined time spans. The drug can be present in a solid state (e.g., in the form of tablets) or in a liquid state (e.g., as a juice) or in a gaseous state or in a mixed form (e.g., as a gel capsule or as an aerosol or as an ointment). A pure substance, a solids mixture, a solution, a suspension (e.g., an emulsion or an aerosol) or the like can be concerned.

The term “capturing intake” means that a check is made as to whether the patient at least took action to take a portion of drug and/or whether said patient actually took a portion of drug. The term “attempted intake” is understood to mean measures taken by a person in order to prepare for an intake of a portion of drug. A typical example is the removal of a portion of drug from a package, for example the removal of a tablet from a blister pack. It is conceivable that preparative measures for the intake of a portion of drug are not personally performed by the patient, but by a physician or by care personnel or by relatives for example. For the present invention, it is irrelevant whether the preparative measure is personally performed by the patient or is performed by another person; the present invention is intended to cover all these possibilities. For simpler presentability, the invention will be mainly described on the basis of the first option (that the patient takes the preparative measures) without any intention to restrict the invention to said option.

It is conceivable that the system according to the invention comprises a drug-intake monitoring unit. In one embodiment of the present invention, said monitoring unit registers whether and when the patient has made preparations for the intake of a portion of drug from a device for storing the drug and/or whether and when the patient has taken a portion of drug. It is conceivable that, before the patient can remove a portion of drug from a storage device, for example by pressing a key or by presenting a biometric feature (e.g., the finger for capture of a fingerprint as part of fingerprint recognition), said patient must signal a wish to remove a portion of drug. In such a case, the monitoring unit registers the action of the patient that is to result in a removal of a portion of drug. However, it is also conceivable that the monitoring unit registers the actual removal of a portion of drug. It is, for example, conceivable that an electrically conductive path is interrupted by a portion of drug being pressed out of a blister pack; this interruption can be captured by an electronic circuit (see, for example, WO9604881A1 or DE19516076A1).

In another embodiment of the present invention, the monitoring unit is designed such that it registers the actual intake of a portion of drug by the patient. The system sold commercially by AiCure can be mentioned here by way of example. In the case of said AiCure system, the actual intake of a portion of drug is tracked by a smartphone app with the aid of the smartphone camera. Image analysis and image recognition algorithms ensure that the portion of drug and the face of the patient are identified. Further identified is mouth-placement and swallowing of the portion of drug by the patient.

In a preferred embodiment, the monitoring unit is designed such that it reminds the patient (or else the care personnel or some other person) of an upcoming intake of a portion of drug; for example, acoustically (e.g., by means of a beep or a voice message), visually (e.g., by flashing of a small light or a text message) and/or tactilely (e.g., by means of vibration). The reminder can occur at defined time points, especially when the patient is to take a portion of drug at defined time points. However, it is also conceivable that the patient is prompted to take one or more portions of drug because a defined event has occurred, for example the exceeding of a threshold of a physiological parameter or the reaching of a physiological state characterized by multiple defined values or value ranges of physiological parameters.

In the data integration unit, there is convergence of all the data from a plurality of sources about a plurality of persons (patients). The data preferably include an unambiguous identifier in order to be able to assign the data to a particular person. The data integration unit is configured such that it receives data from the data sources and performs an analysis as to which person incoming data must be assigned. It is conceivable that the data integration unit performs an analysis as to from which sender the data originate; possibly, an unambiguous assignment to a patient can be performed with reference to the sender (e.g., on the basis of an IP address of, for example, the self-assessment unit or a sensor unit). Possibly, a patient to whom the data belong can be unambiguously indicated on the basis of an unambiguous identifier which is, for example, stored in the header.

It is conceivable that data are transmitted to the data integration unit in encrypted form and/or in signed form. The data integration unit can be configured such that it decrypts encrypted data and/or checks the signature in the case of signed data.

The data integration unit can be configured such that it checks incoming data for readability and/or completeness.

The data integration unit can be configured such that it transmits an error message to an administrator in the event of data which cannot be decrypted and/or data which have an unknown/wrong signature and/or in the event of incomplete data and/or unreadable data.

The data integration unit is configured such that it transmits incoming data, after a processing step if necessary, to a data synchronization unit.

The data synchronization unit is configured such that it synchronizes various data relating to a particular person. The term “synchronization” refers to the temporal alignment of processes. A synchronization ensures that processes proceed simultaneously (synchronously) or in a manner such that they are arranged temporally in a certain order. Synchronization by the data synchronization unit concerns positioning the various data relating to one person in relation to one another in terms of time. The acquired data provide information about events which took place at certain time points or within certain time spans. Synchronization involves sorting the data according to the time points and time spans of the underlying events and positioning them on a common time axis.

It is conceivable that the data transmitted from a data source have one or more timestamps. Timestamps are used for assigning an unambiguous time point or a time span to an event. For example, if the heart rate is ascertained by means of a sensor, it is important to know when the person had the ascertained heart rate in order to relate the heart rate to further physiological parameters which were present beforehand, at the same time point or in the same time span and/or afterwards. In this way, it is possible to identify correlations and possibly causes of physiological states. As timestamp for sensor data, it is possible to use, for example, the start or the end of a measurement. As timestamp for laboratory data, it is possible to use, for example, the time point of sampling. As timestamp for self-assessment data, it is possible to use the time point of a question being asked, the time point of that event to which a question refers and/or the time point of a question being answered.

It is conceivable that the self-assessment unit and/or the laboratory data acquisition unit and/or one or more sensor units used and/or individual sensors or all sensors have a timepiece for providing data with one or more timestamps. It is conceivable that multiple units are synchronized with one another so that they have a synchronous system time. For example, such a synchronization can be achieved by one unit transmitting to one or more other units at defined time points a signal that the time point in question has been reached. The units to which the signal has been transmitted consequently adjust their system time to the system time of the transmitting unit.

It is also conceivable that two sensors capture the same physiological parameter or a similar physiological parameter. By superposing the measurement values and temporal shift of the measurement values relative to one another until coverage is reached by the measurement values, it is possible to temporally synchronize the measurement values.

After all transmitted data have been temporally synchronized, the data are stored in a data memory. There, they can, for example, be retrieved by a data display unit and/or a data analysis unit. The synchronized data show the temporal course of physiological parameters and of the self-assessment along a common time axis. The interplay of physiological states is identifiable as a result. For example, it is possible to investigate what effect the intake of a portion of drug has on physiological parameters of the patient. It is possible to investigate how the health status of a patient improves or deteriorates in the course of time and/or in the context of a therapy. It is possible to investigate whether an increased activity of the patient has a positive effect on laboratory values and/or physiological parameters and/or the state of health of the patient. It is possible to investigate whether the self-assessment correlates with one or more physiological parameters. In the event of a deterioration in the health status, which can be identified from the self-assessment and/or laboratory values and/or physiological parameters, a rapid intervention can be made in order to help the patient. Therapeutic measures can be attuned to changing physiological parameters and/or laboratory values and/or the self-assessed health status.

The present invention can be used as part of a therapy or preventively or as part of a clinical study. Preferably, it is used for therapeutically accompanying patients for whom a heart failure has been diagnosed. Very particularly preferably, it is used for accompanying patients who are suffering from HFpEF, very particularly preferably in a hospital and/or after discharge from a hospital.

The invention is more particularly elucidated below with reference to figures and examples, without wishing to restrict the invention to the features and combinations of features that are specified in the figures and examples.

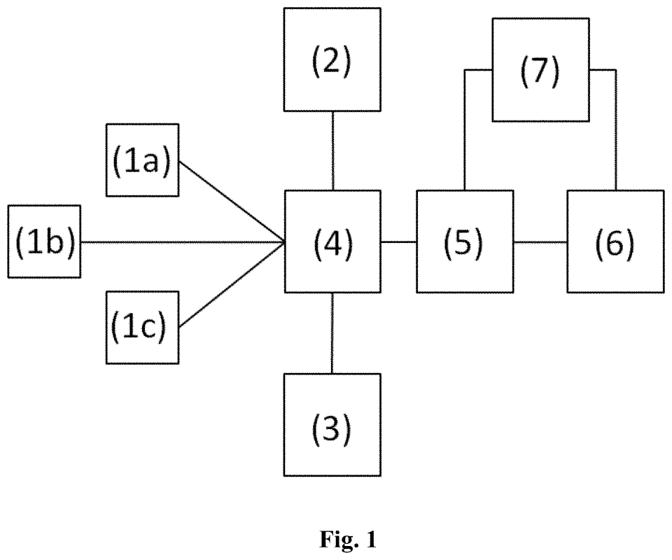

FIG. 1 shows schematically one embodiment of the system according to the invention.

The system comprises sensors (1a, 1b, 1c) for monitoring physiological parameters of a person, a self-assessment unit (2), a laboratory data acquisition unit (3), a data integration unit (4), a data synchronization unit (5), a data memory (6) and a data display and/or data analysis unit (7).

The sensors (1a, 1b, 1c) are configured and connected to the data integration unit (4) such that they transmit permanently captured measurement values relating to physiological parameters of a person to the data integration unit (4). The self-assessment unit (2) is configured and connected to the data integration unit (4) such that it transmits information relating to the assessment by the person about his/her health status to the data integration unit (4) at regular or irregular time intervals. The laboratory data acquisition unit (3) is configured and connected to the data integration unit (4) such that it transmits further data relating to the health status of the person to the data integration unit (4) at regular or irregular time intervals. The data integration unit (4) is configured such that it receives data from the sensors (1a, 1b, 1c), from the self-assessment unit (2) and from the laboratory data acquisition unit (3) and assigns them to the corresponding person. The data integration unit (4) is configured and connected to the data synchronization unit (5) such that it transmits the data assigned to the person to the data synchronization unit (5). The data synchronization unit (5) is configured such that it temporally synchronizes the data transmitted from the data integration unit (4). The data synchronization unit (5) is configured and connected to the data memory (6) such that it transmits the person-assigned and temporally synchronized data to the data memory (6). The data memory (6) is configured such that it receives and stores the data transmitted from the data synchronization unit (5). The data synchronization unit (5) is configured and connected to the data display and/or data analysis unit (7) such that it transmits the person-assigned and temporally synchronized data to the data display and/or data analysis unit (7). The data display and/or data analysis unit (7) is configured such that it displays the data transmitted from the data synchronization unit (5) on a display unit and/or introduces them to an analysis. The data display and/or data analysis unit (7) is furthermore configured and connected to the database (6) such that it reads the person-assigned and temporally synchronized data from the database (6) and displays them on a display unit and/or introduces them to an analysis.

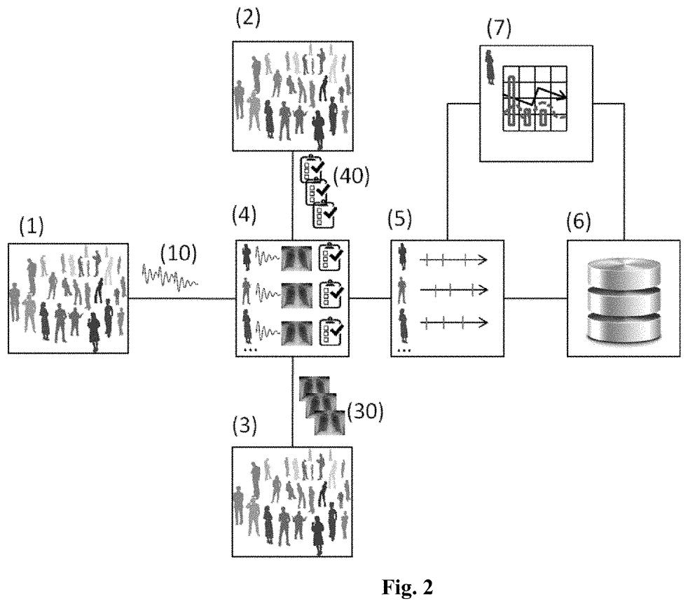

FIG. 2 shows schematically a further embodiment of the system according to the invention.

In FIG. 2, the graphic element with reference sign (1) represents a multiplicity of sensors which capture measurement values for a multiplicity of persons. The persons can use the same sensors or different sensors. The persons can use the same number of sensors or different numbers of sensors. All the sensors are configured such that they transmit measurement values (10) to a data integration unit. The one data integration unit is represented by reference sign (4). The graphic element with reference sign (2) represents a multiplicity of self-assessment units used by a multiplicity of persons for collecting information relating to subjective health status. Usually, each person uses one personal self-assessment unit. However, it is also conceivable that multiple persons share one self-assessment unit. Further conceivable is that one person uses multiple self-assessment units; for example, one at work and another at home. The self-assessment units are configured such that they transmit information (40) relating to the respective subjective health status of the persons to the data integration unit. The graphic element with reference sign (3) represents at least one laboratory data acquisition unit, by means of which information (30) relating to health status are acquired from a multiplicity of persons and transmitted to the data integration unit. The data integration unit is configured such that it analyzes all incoming data (information) for labels of that person whose health status they provide information about and assigns them to said person. The data assigned to the individual persons are transmitted from the data integration unit to a data synchronization unit. The data synchronisation unit is represented in FIG. 2 by the graphic element with reference sign (5). Usually, the data which have been transmitted from the data integration unit to a data synchronization unit have timestamps. On the basis of these timestamps and/or other information, the data are temporally synchronized, i.e., temporally arranged and positioned on a time axis for each individual person. The data synchronization unit is configured such that it stores data in one or more databases and/or transmits data to one or more data display and/or data analysis units. The graphic element with reference sign (6) represents one or more data memories for storing the data which are assigned to the individual persons and temporally synchronized. The graphic element with reference sign (7) represents one or more data display and/or data analysis units. The at least one data display and/or data analysis unit is configured such that it displays the data transmitted from the data synchronization unit or the data read from the at least one data memory on a display unit and/or introduces them to an analysis.

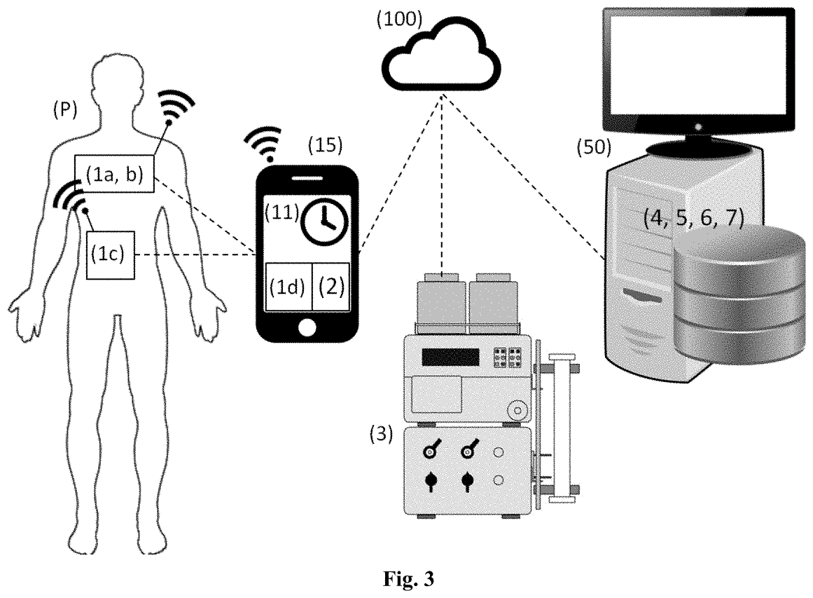

FIG. 3 shows schematically a further embodiment of the system according to the invention from the perspective of a patient (P). The patient (P) has four sensors (1a, 1b, 1c, 1d) which permanently capture measurement values. Two sensors (1a, 1b) are combined in one sensor unit which is worn in the body of the patient (P); for example, a sensor unit which captures measurement values from which an electrocardiogram, respiratory rate, body temperature and/or body fluid status can be ascertained. A further sensor (1c) is worn on the body of the patient (P), for example an activity tracker. The sensors (1a, 1b, 1c) transmit the captured measurement values, for example via radio communication (e.g., via a Bluetooth connection), to the person's smartphone (15). The smartphone (15) receives the measurement values of the sensors (1a, 1b, 1c). The smartphone (15) is equipped with a further sensor (1d), for example an activity tracker. The smartphone (15) furthermore acts as a self-assessment unit (2), i.e., it is configured such that it prompts the patient (P) at defined time points to input information about his/her subjective health status into the smartphone (15). The smartphone (15) comprises a timepiece (11). The smartphone (15) is configured such that it provides incoming measurement values from the sensors (1a, 1b, 1c) and the information relating to the self-assessment of the patient (P) with timestamps and provides them with an individual identifier, on the basis of which the identity of the patient (P) can be ascertained. The smartphone (15) is configured such that it transmits all data (information) which have been captured and provided with individual identifiers and timestamps to a network memory (100), represented by a cloud, at defined time points via a cellular network and/or a contactless network (WiFi network). A laboratory data acquisition unit (3) is configured such that it provides laboratory data relating to the patient (P) with an individual identifier and a timestamp (e.g., time point of sampling and/or examining the patient) and likewise transmits them to the network memory (100). A computer system (50) comprising data memory, display means and calculation unit is likewise connected to the network memory (100) via a network and can access the data in the network memory (100). The computer system (50) is configured such that it can execute the functionalities of data integration unit (4), data synchronization unit (5), data memory (6) and data display and/or data analysis unit (7).

1. A system for monitoring a plurality of persons who are suffering from heart failure, comprising:
sensors for monitoring physiological parameters of a person;
a self-assessment unit;
a data integration unit;
a data synchronization unit;
a data memory; and
a laboratory data acquisition unit,
wherein the sensors, the self-assessment unit and the laboratory data acquisition unit are connected to the data integration unit via a network,
wherein the sensors, the self-assessment unit and the laboratory data acquisition unit are configured to transmit data to the data integration unit via the network,
wherein the data integration unit is configured to receive data from the sensors, the self-assessment unit and the laboratory data acquisition unit, link the received data together based on the person and transmit the linked data to a data synchronization unit,
wherein the data synchronization unit is configured to receive the linked data from the data integration unit, temporally synchronize the received linked data, and transmit the temporally synchronized data to the data memory, and
wherein the data memory is configured to store the temporally synchronized data.

2. The system of claim 1, wherein the data integration unit is configured to:
receive measurement values relating to a plurality of persons from a plurality of sensors; and
assign, based on individual identifiers of the plurality of persons, the measurement values to the persons whose health status the measurement values provide information about.

3. The system of claim 1, wherein the data synchronization unit is configured to:
temporally sort the data received from the data integration unit based on timestamps; and
position the temporally sorted data on a time axis for each person.

4. The system of claim 1, further comprising a data display unit, configured to display the temporally synchronized data for each person.

5. The system of claim 1, further comprising a data analysis unit configured to search for correlations in the temporally synchronized data and transmit results of the analysis to a data display unit for display.

6. The system of claim 1, wherein the sensors are configured to be arranged to capture measurement values from which the following parameters of the person can be ascertained: physical activity, electrocardiogram, respiratory rate, body temperature, and body fluid status.

7. The system of claim 1, further comprising a drug-intake monitoring unit configured to register an intake of a portion of drug by the person or an attempted intake, generate a file relating to the intake or the attempted intake, provide the file with an unambiguous identifier relating to the person, provide the file with a timestamp providing information about when the intake or the attempted intake took place, and transmit the file to the data integration unit.

8. The system of claim 7, comprising a data analysis unit configured to search for correlations between the intake of a portion of drug and the temporally synchronized data relating to a health status of the person and transmit the searched correlations to a data display unit for display.

9. A method for monitoring a plurality of persons who are suffering from heart failure, comprising:
monitoring physiological parameters of a person using sensors, the monitoring comprising permanently capturing sensor data using the sensors;
ascertaining self-assessment data from the person;
ascertaining laboratory data relating to the person;
transmitting the sensor data, the self-assessment data, and the laboratory data to a data integration unit via a network;
linking, by the data integration unit, the sensor data, the self-assessment data and the laboratory data based on the person;
transmitting the linked data to a data synchronization unit;
temporally synchronizing, by the data synchronization unit, the linked sensor data, self-assessment data and laboratory data;
transmitting the temporally synchronized data to a data memory; and
storing the temporally synchronized data on the data memory.

10. The method of claim 9, further comprising:
providing the data captured by the sensors, the data ascertained by the self-assessment unit, and the data acquired by the laboratory data acquisition unit with timestamps at the time points at which the sensor data were captured, at which a self-assessment was performed, at which a sample introduced to a laboratory investigation was taken, or at which a medical specialist examination was performed; and
temporally synchronizing the data based on the timestamps.

11. The method of claim 9, further comprising:
providing the data captured by the sensors the data ascertained by the self-assessment unit, and the data acquired by the laboratory data acquisition unit with an unambiguous identifier, through which the person whose health status the data provide information about can be ascertained; and
assigning the data to the person based on the unambiguous identifier.

12. The method of claim 9, further comprising:
registering an intake of one or more portions of drug by the person or an attempted intake;
generating a file comprising information relating to the number and the nature of the one or more portions of drug;
providing the file with an unambiguous identifier relating to the person;
providing the file with a timestamp which provides information about the time point of the intake or the attempted intake; and
transmitting the file to the data integration unit.

13. The method of claim 9, further comprising:
displaying the temporally synchronized data.

14. The method of claim 10, further comprising:
analyzing the temporally synchronized data with respect to:
an improvement/deterioration in the health status of the person,
an effect of drugs on physiological parameters of the person,
correlations between different physiological parameters, and
correlations between data from the self-assessment and physiological parameters;
displaying the results of analyzing the temporally synchronized data; and
initiating measures in response to determining a deterioration in the health status of the person.

15. (canceled)
